(19) United States
(12) Patent Application Publication
(10) Pub. No.: US 2008/0221600 A1
(43) Pub. Date: Sep. 11, 2008

(54) ISOLATION DEVICES FOR THE TREATMENT OF ANEURYSMS

(76) Inventors: Martin S. Dieck, Cupertino, CA (US); Brian B. Martin, Felton, CA (US)

Correspondence Address: Nfocus Neuromedical Inc.,c/o Levine Bagade Han LLP, 2483 East Bayshore Road, Suite 100, Palo Alto, CA, 94303, US

(21) Appl. No.: 11/840,718

(22) Filed: Aug. 17, 2007

Related U.S. Application Data
(60) Provisional application No. 60/822,745, filed on Aug. 17, 2006.

(51) Int. Cl. A61B 17/08

(52) U.S. Cl. 606/157

(57) ABSTRACT

Device, systems and methods are provided to isolate aneurysms, particularly at bifurcations, while maintaining adequate blood flow through nearby vessels. These devices are deliverable to a desired target area and maintain position in a desired orientation so as to occlude flow in some aspect while allowing flow in others. In addition, devices, systems and methods are provided to occlude blood vessels, such as endoleaks, to improve the isolation of aneurysms.

### CROSS-REFERENCE TO RELATED APPLICATIONS

This application is a non-provisional of U.S. Provisional Application No. 60/822,745 filed on Aug. 17, 2006 entitled ISOLATION DEVICES FOR THE TREATMENT OF ANEURYSMS. The entirety of which is hereby incorporated by reference.

### BACKGROUND OF THE INVENTION

The term aneurysm refers to any localized widening or outpouching of an artery, a vein, or the heart. All aneurysms are potentially dangerous since the wall of the dilated portion of the involved vessel can become weakened and may possibly rupture. One of the most common types of aneurysms involve the aorta, the large vessel that carries oxygen-containing blood away from the heart. In particular, aneurysms most commonly develop in the abdominal portion of the aorta and are designated abdominal aortic aneurysms (AAA). Abdominal aortic aneurysms are most common in men over the age of 60.

There are approximately 40,000 patients undergoing elective repair of abdominal aortic aneurysm in the United States each year. In spite of that, approximately 15,000 patients die from ruptured aneurysm, making aneurysm rupture the 13th leading cause of death in the United States each year. This cause of premature death is entirely preventable providing that patients with abdominal aortic aneurysm can be diagnosed prior to rupture and undergo safe elective repair of the abdominal aortic aneurysm. Elective repair of abdominal aortic aneurysm has matured over the 45-year interval since the first direct surgical repair of abdominal aortic aneurysm was performed. Conventional open surgical repair of abdominal aortic aneurysm has often been replaced by endovascular repair which involves a minimally invasive technique. Endovascular repair of abdominal aortic aneurysm utilizes access to the vascular system, through the femoral artery, to place a graft of appropriate design in the abdominal aorta in order to remove the aneurysm from the pathway of bloodflow and thus reduce the risk of rupture.

Another type of aneurysm is a brain aneurysm. Brain aneurysms are widened areas of arteries or veins within the brain itself. These may be caused by head injury, an inherited (congenital) malformation of the vessels, high blood pressure, or atherosclerosis. A common type of brain aneurysm is known as a berry aneurysm. Berry aneurysms are small, berry-shaped outpouchings of the main arteries that supply the brain, and are particularly dangerous since they are susceptible to rupture, leading to often fatal bleeding within the brain. Brain aneurysms can occur at any age but are more common in adults than in children.

Currently, a variety of methods are used to treat brain aneurysms. Neuroradiological (catheter-based or endovascular) nonsurgical procedures include: (i) placement of metallic (e.g., titanium) microcoils or a “glue” (or similar composite) in the lumen of the brain aneurysm (in order to slow the flow of blood in the lumen, encouraging the aneurysm to clot off (be excluded) from the main artery and hopefully shrink; (ii) placement of a balloon with or without microcoils in the parent artery feeding the brain aneurysm (again, with the intention of stopping the flow of blood into the brain aneurysm lumen, encouraging it to clot off and hopefully shrink); (iii) insertion of a stent across the aneurysmal part of the artery to effectively cut off blood supply to the brain aneurysm, or to allow coiling through openings in the stent, without stopping blood flow across the open stent; and (iv) a combination of the previous three procedures. These procedures provide many advantages including allowing access to aneurysms that are difficult to access surgically.

However, there are still many deficiencies in these treatments. Covered stents designed to cover aneurysms face the challenge of effectively covering the aneurysm while not occluding nearby blood vessels. If the covering is too long, the nearby blood vessels may be occluded creating additional potential harm for the patient. And, conventional stents, both covered and uncovered, have difficulty targeting aneurysms located at a bifurcation or trifurcation. A berry aneurysm located at a bifurcation is illustrated in FIG. 1. The aneurysm A is located near the end of a trunk T, between two distal branches B. Blood flowing through the trunk T continues through the branches B but also flows into the aneurysm A, creating pressures and accumulation which may lead to rupture. Typically, such aneurysms are accessed via the trunk T creating difficulty accessing both distal branches B. Current attempts utilize bifurcated stents with multiple arms and multiple wires to traverse the blood vessels resulting in very complex systems. Consequently, improved devices are desired to isolate aneurysms, particularly at bifurcations, while maintaining adequate blood flow through nearby vessels. These devices should be relatively easy to produce, deliver to a desired target area, and maintain position in a desired orientation so as to occlude flow in some aspect while allowing flow in others. At least some of these objectives will be met by the present invention.

In the case of stented abdominal aneurysms, at least 30% of such stented abdominal aortic aneurysms have endoleaks. FIG. 2 illustrates an abdominal aortic aneurysm AAA having a stent 2 placed therein to isolate the aneurysm AAA. Endoleaks E are shown extending from the aneurysm AAA. Many of these endoleaks E are caused by collateral flow from the mesenteric (3-4 mm) arteries and the lumbar (2-3 mm) arteries. In some cases, though less commonly, such endoleaks are caused by collateral flow from the renal (5-6 mm) arteries. Such endoleaks E allow blood to flow into the aneurysm increasing the risk of rupture. Consequently, improved devices are desired to isolate such aneurysms while reducing the incidence of endoleaks. At least some of these objectives will be met by the present invention.

### SUMMARY OF THE INVENTION

The description, objects and advantages of the present invention will become apparent from the detailed description to follow, together with the accompanying drawings.

### BRIEF DESCRIPTION OF THE DRAWINGS

FIG. 1 illustrates a berry aneurysm located at a bifurcation of a blood vessel.

FIG. 2 illustrates an abdominal aortic aneurysm having a conventional stent placed therein.

FIG. 3 illustrates an embodiment of an isolation device of the present invention having an occluder.

FIG. 4 illustrates an isolation device having the form of a coil.

FIGS. 5A-5B illustrate an isolation device constructed from a sheet.

FIG. 6 illustrates an isolation device wherein the occluder comprises a diverter.

FIG. 7 illustrates an isolation device having a conical shape.

FIG. 8 illustrates an isolation device having a body configured for positioning within a neck of an aneurysm.

FIG. 9 illustrates an embodiment of an isolation device having a sack which may extend into the aneurysm.

FIG. 10 illustrates an isolation device having a portion constructed so as to anchor within the trunk of the blood vessel.

FIG. 11 illustrates an embodiment of an isolation device having an occluder comprising struts.

FIGS. 12-13 illustrate isolation devices comprising a body having a single end.

FIGS. 14-15 illustrate isolation devices comprising a body having a ball shape.

FIG. 16A-16C illustrate a method of constructing a ball shaped isolation device.

FIG. 17A-17B illustrate a ball shaped isolation device having articulating struts.

FIGS. 18A-18C illustrate a ball shaped isolation device formed from individual coils.

FIGS. 19A-19C illustrate a ball shaped isolation device formed from individual coils including a cover.

FIGS. 20A-20C illustrate a method of delivery of the isolation device of FIGS. 18A-18C.

FIG. 21 illustrates an abdominal aortic aneurysm having endoleaks occluded by isolation devices of the present invention.

FIGS. 22A-22C illustrates an isolation device of the present invention having an occluder.

FIGS. 23A-23C illustrates an isolation device having a body in the form of a coil.

FIGS. 24A-24C illustrate an isolation device constructed from a sheet.

FIG. 25 illustrates an isolation device having an occluder comprising fibers.

FIG. 26 illustrates an isolation device having an occluder comprising a biocompatible filler.

FIGS. 27A-27B illustrate an isolation device having an occluder comprising a sack.

FIGS. 28A-28B illustrate an isolation device having an occluder comprising a valve.

FIGS. 29A-29C illustrate an isolation device having an occluder comprising a flap.

FIGS. 30, 31, 32 illustrate various embodiments of isolation devices having a conical shape.

FIG. 33A-33B illustrate an isolation device having a conical shape and an occluder comprising a flap.

FIG. 34A-34B illustrate an isolation device comprising a pair of conical shaped bodies.

FIG. 35 illustrates a variety of methods of incorporating radiopaque material into the body of an isolation device.

FIG. 36 illustrates a method of joining two types of material.

FIG. 37A-37B illustrates a push-style delivery system.

FIG. 38 illustrates a pull-style delivery system.

FIG. 39A-39C illustrates a sheath-style delivery system.

FIG. 40A-40C illustrate a balloon expandable delivery system.

FIG. 41A-41B illustrate an isolation device comprising a shape memory element coupled with a portion of material.

FIG. 42A-42D illustrate an isolation device comprising a coil having a polymeric covering.

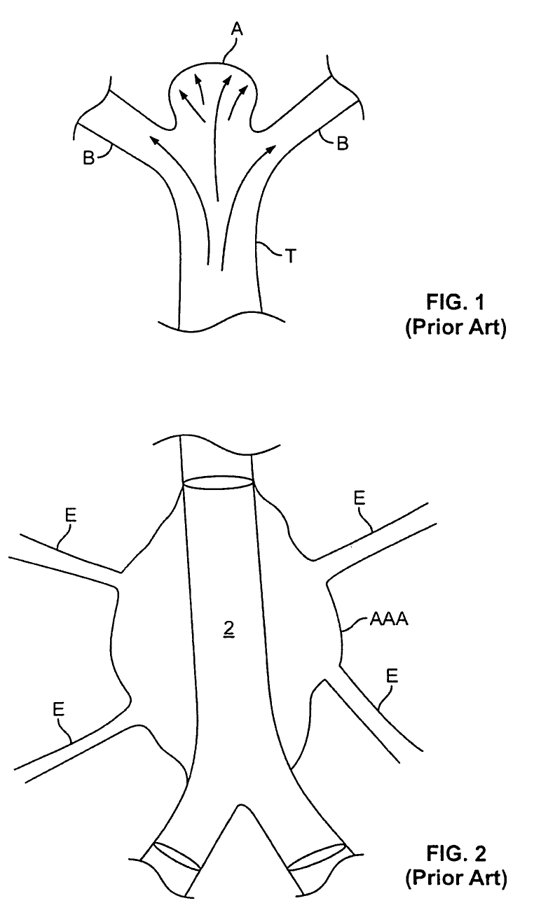

### DETAILED DESCRIPTION OF THE INVENTION

Devices for Treatment of Berry Aneurysms

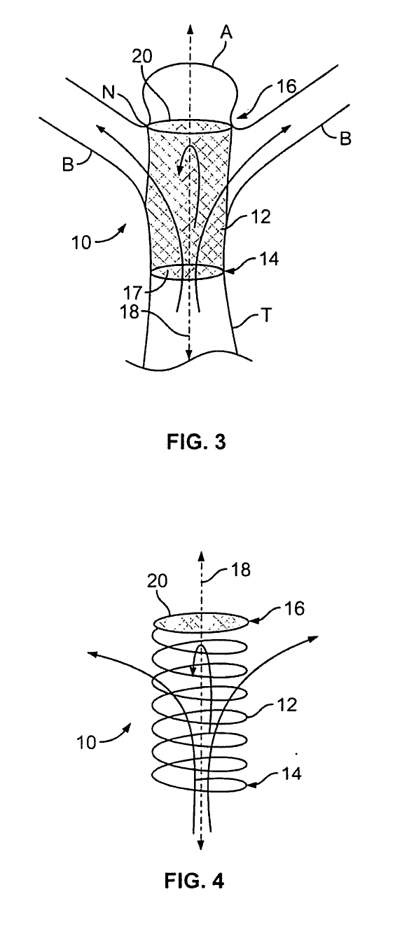

A variety of isolation devices are provided for treating berry aneurysms, particularly berry aneurysms located at bifurcations or other branched vessels. An embodiment of such an isolation device is illustrated in FIG. 3. Here, the isolation device 10 comprises a body 12 having a first; end 14, a second end 16 and a lumen 17 extending therethrough along a longitudinal axis 18. The isolation device 10 also includes an occluder 20 which occludes blood flow in at least one direction. In this embodiment, the occluder 20 is located near the second end 16 occluding blood flow along the longitudinal axis 14, so as to act as an axial occluder, and diverting flow away from the longitudinal axis 14.

The body 12 may have any suitable shape or design, such as a cylindrical shape as shown. Further, the body 12 may be comprised of any suitable construction, such as braid, mesh, lattice, coil, struts or other construction. The body 12 shown in FIG. 3 has a wire braid construction. Likewise, the occluder 20 may have any suitable shape, design or construction. For example, the occluder 20 may be comprised of a solid sheet, a sheet having openings, a mesh, a lattice, struts, threads, fibers, filaments, a biocompatible filler or adhesive, or other suitable material. The occluder 20 shown in FIG. 3 comprises a solid sheet extending across the second end 16.

The isolation device 10 is positioned within the trunk T of the bifurcated blood vessel so that the second end 16 is disposed near the aneurysm A, preferably within, against or near a neck N of the aneurysm A. Thus, blood flowing through the trunk T is able to flow through the device 10, entering through the first end 14 and exiting radially through the sides of the body 12 to the distal branches B. Flow is resisted through the second end 16 by the occluder 20. Thus, the aneurysm A is isolated from the blood vessel without restricting flow through the trunk T or distal branches B. In some embodiments, the body 12 has varied construction along its length to facilitate radial flow through the sides of the body 12. For example, the braid, mesh or lattice may have larger openings in specific areas to facilitate flow therethrough.

FIG. 4 illustrates another embodiment of an isolation device 10. Here the body 12 has the form of a coil. Again the body 12 has a first end 14 and a second end 16. The device 10 also includes an occluder 20 located near the second end 16. Thus, flow entering the first end 14 is resisted through the second end 16 by the occluder 20 but is allowed to flow radially outwardly through the sides of the body 12. Again, the body 12 may have varied construction along its length to facilitate radial flow through the sides of the body 12. For example, the pitch of the coil may be increased in specific areas to facilitate flow therethrough.

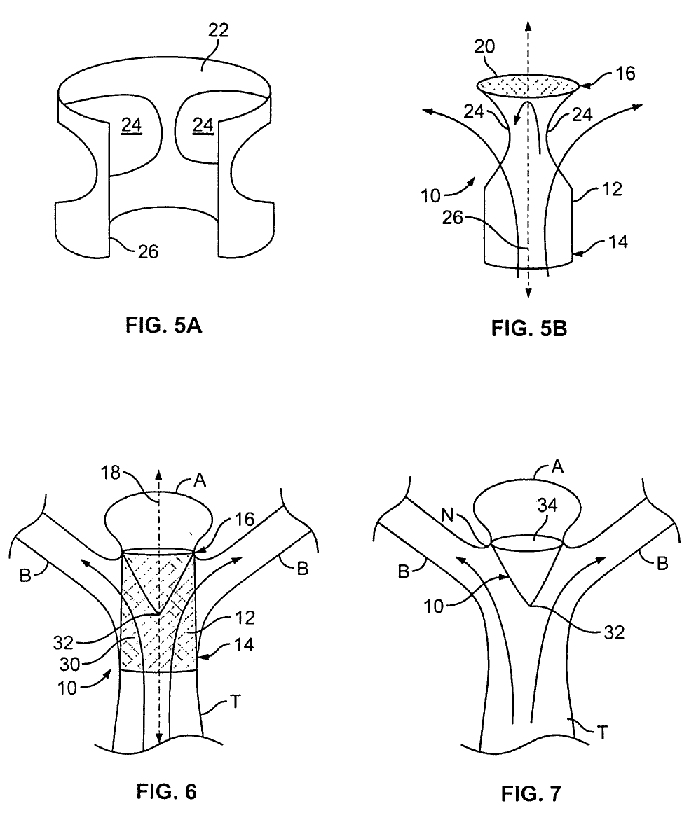

FIGS. 5A-5B illustrate an isolation device 10 constructed from a sheet 22. FIG. 5A illustrates a sheet 22 having at least one opening 24. The sheet 22 is joined, coupled or overlapped along an edge 26 so as to form the body 12 of the device 10 having a cylindrical shape. FIG. 5B illustrates the device 10 having a body 12 constructed as in FIG. 5A and all occluder 20 disposed near the second end 16. Thus, blood flowing through the first end 14 is resisted at the second end 16 by the occluder 20 but is allowed to flow radially outwardly through the at least one opening 24.

Referring to FIG. 6, in some embodiments the occluder comprises a diverter 30. The diverter 30 diverts flow, typically within the body 12 of the isolation device 10 so as to redirect flow from along the longitudinal axis to a radially outwardly direction. The diverter 30 illustrated in FIG. 6 has a conical shape wherein a tip 32 of the conical diverter 30 extends into the body 12 along the longitudinal axis 18 and faces the first end 14. Thus, blood flow entering the first end 14 is diverted radially outwardly through the sides of the body 12 to the distal branches B by the diverter 30. Consequently, blood does not enter the aneurysm A. It may be appreciated that the diverter 30 may have any suitable shape including flat, stepped, curved, radiused, convex and concave, to name a few.

In some embodiments, as shown in FIG. 7, the body 12 of the isolation device 10 acts as a diverter. Here, the body 12 has a base 34 is positioned within, against or near the neck N of the aneurysm A and a conical tip 32 facing the trunk T. Thus, blood flowing through the trunk T is diverted into the distal branches B.

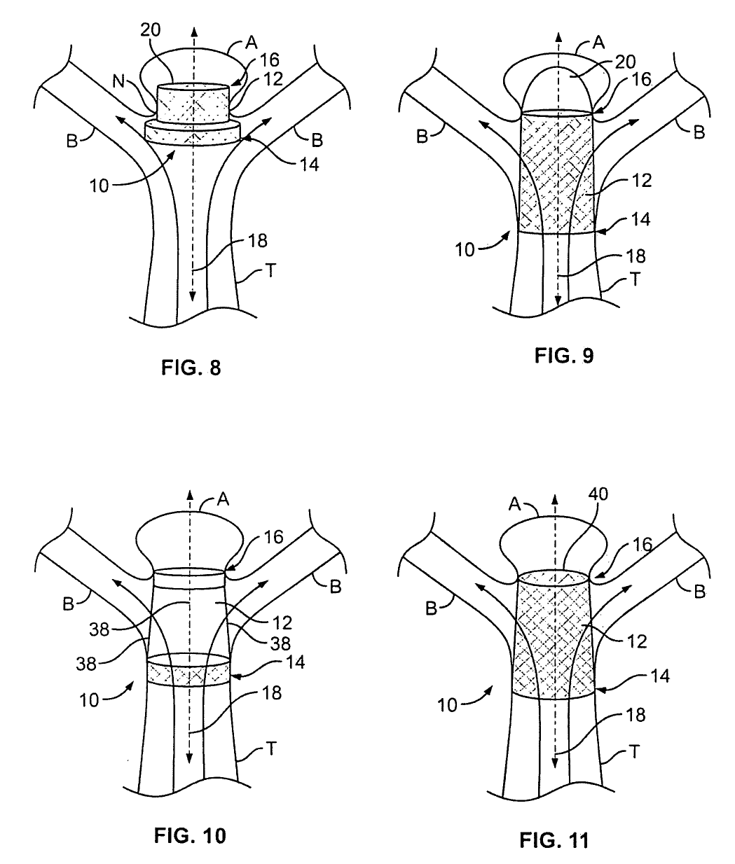

FIG. 8 illustrates an embodiment of an isolation device 10 comprising a body 12 having a first end 14, a second end 16 and a longitudinal axis 18 therethrough. The body 12 is configured so that the first end 14 resides outside of the neck N of the aneurysm A and is secured against the neck N, such as by virtue of a wider dimension or lip which is prevented from passing through the neck N. The body 12 extends through the neck N so that the second end 16 resides within the aneurysm A. An occluder 20 may be disposed near the second end 16, as shown, near the first end 14 or anywhere therebetween to resist blood flow from entering the aneurysm A. Thus, blood flowing through the trunk T of the vessel freely flows to the distal branches B without significantly passing through the isolation device 10.

FIG. 9 illustrates an embodiment of an isolation device 10 comprising a body 12 having a first end 14, a second end 16 and a longitudinal axis 18 therethrough. Here, the body 12 is configured similar to the embodiment of FIG. 3. However, here the occluder 20 comprises a bag or sack of a flexible material which may extend into the aneurysm A.

FIG. 10 illustrates an embodiment of an isolation device 10 comprising a body 12 having a first end 14, a second end 16 and a longitudinal axis 18 therethrough. In this embodiment, the first end 14 is constructed so as to act as an anchor within the trunk T. For example, the first end 14 may have a braided construction which provides radial force. In addition, the first end 14 may include anchors, such as hooks, loops, or spikes which engage a wall of the blood vessel. The second end 16 is constructed so as to atraumatically reside within, against or near the neck N of the aneurysm A. Thus, the second end 16 provides less radial force. The body 12 extending between the ends 14, 16 may have any suitable construction, such as a braid, mesh, lattice, coil, struts, to name a few. In this embodiment, the body 12 comprises struts 38 extending between the ends 14, 16. Thus, blood flow entering the first end 14 may flow radially outwardly between the struts 38 to the distal branches B.

FIG. 11 illustrates an embodiment of an isolation device 10 comprising a body 12 having a first end 14, a second end 16 and a longitudinal axis 18 therethrough. Here, the body 12 is configured similar to the embodiment of FIG. 3. However, in this embodiment the occluder 20 comprises struts 40 extending across the second end 16. The struts 40 have a denser configuration than the body 12 so as to reduce flow therethrough.

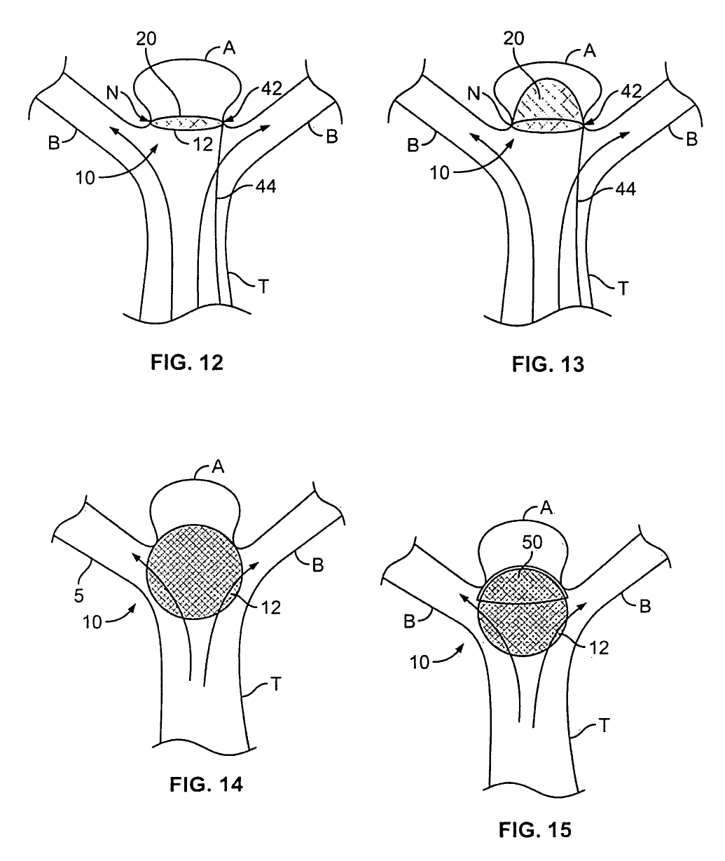

FIG. 12 illustrates an embodiment of an isolation device 10 comprising a body 12 having a single end 42. The end 42 is positionable within, against or near the neck N of the aneurysm A as shown, with the use of a guide 44. In this embodiment, an occluder 20 extends across the end 42 to prevent flow into the aneurysm A. To assist in holding the end 42 near the neck N, the end 42 may be radiofrequency (rf) welded to the neck N area.

FIG. 13 illustrates another embodiment of an isolation device 10 comprising a body 12 having a single end 42. Again, the end 42 is positionable within, against or near the neck N of the aneurysm A as shown, with the use of a guide 44. In this embodiment, an occluder 20 has the shape of a bag or sack extending into the aneurysm A. Such extension into the aneurysm A may reduce any risk of dislodgement, particularly if the occluder 20 has some rigidity. To assist in holding the end 42 near the neck N, the end 42 may be radiofrequency (rf) welded to the neck N area.

FIG. 14 illustrates another embodiment of an isolation device 10. Here, the isolation device 10 comprises a body 12 having a ball shape which includes round, spherical, elliptical, oval and egg-shaped. Thus, the ball shaped body 12 may be disposed within the intersection of the trunk T, distal branches B and aneurysm A. The ball shape allows the body 12 to reside within the intersection without the need for anchoring within a specific vessel. Optionally, the device 10 may be slightly oversized within the intersection to assist in its stability and security. The body 12 may be comprised of any suitable construction, such as braid, mesh, lattice, coil, struts or other construction. Blood flowing through the trunk T enters the body 12 and exits the body 12 through to the distal branches B while flow to the aneurysm A is prevented. This is achieved by varying the density of the construction. For example, a body 12 constructed of mesh may have a denser mesh configuration over the aneurysm A and a looser mesh over the distal branches B. Optionally, the body 12 may include openings or apertures therethrough, such as substantially aligned with the distal branches B or trunk T, so as to allow access or crossing by a catheter. Further, as illustrated in FIG. 15, the device 10 may include a cover 50 which extends over a desired portion of the body 12. The cover 50 may be of any suitable size, shape or material. For example, the cover 50 may be comprised of ePTFE and may cover a portion of the body 12 slightly larger than the neck N of the aneurysm A. Thus, the cover 50 may assist in preventing flow into the aneurysm A.

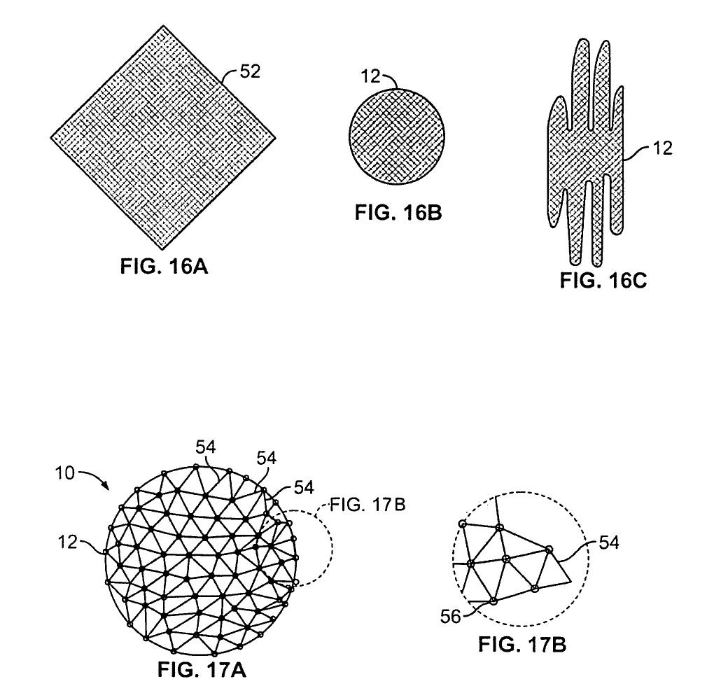

FIGS. 16A-16C illustrate a method of constructing the isolation device 10 of FIG. 14. FIG. 16A illustrates a mesh sheet 52 comprised of a suitable material, such as nitinol wire. The sheet 52 is then formed into a ball-shaped body 12 by wrapping the sheet 52 so that the ends substantially align and the ends are trimmed and laser welded, as illustrated in FIG. 16B. The ball-shaped body 12 may then be compressed, as illustrated in FIG. 16C, for delivery through a delivery catheter.

In some embodiments, the ball-shaped body 12 of the isolation device 10 is comprised of articulating struts 54, as illustrated in FIGS. 17A-17B. FIG. 17A shows the body 12 comprised of such struts 54 and FIG. 17B shows an expanded view of a portion of the body 12 showing the individual struts 54 connected by joints 56 which allow the struts 54 to rotate in relation to each other. Such articulating struts 54 may allow the use of more rigid materials since the struts 54 may rotate in relation to each other to facilitate compression of the device 10 for delivery. Alternatively, the struts 54 may bend or angulate to facilitate compression.

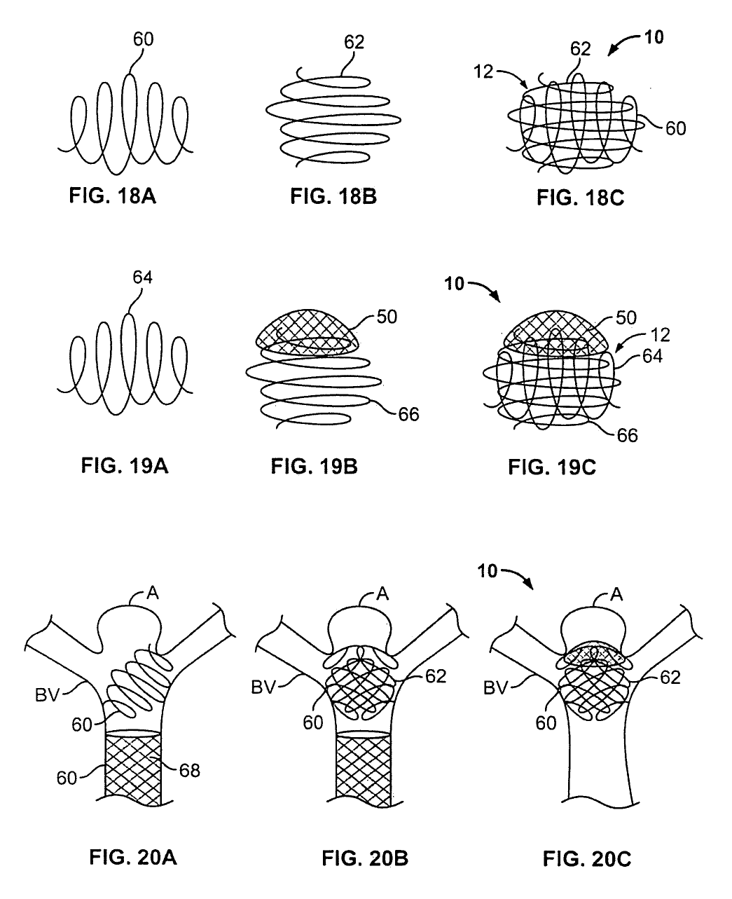

In some embodiments, the isolation device 10 is comprised of separate parts that together form the isolation device 10. For example, referring to FIGS. 18A-18C, an isolation device 10 having a ball-shaped body 12 may be formed from individual coils. FIG. 18A illustrates a first coil 60 positioned horizontally and FIG. 18B illustrates a second coil 62 positioned vertically. In this embodiment, each of the coils 60, 62 vary in diameter, varying from smaller near its ends and larger near its center. FIG. 18C illustrates the combination of the first coil 60 and second coil 62 forming a ball-shaped body 12. By positioning the coils 60, 62 substantially perpendicularly to each other, the larger center of the first coil 60 engages the smaller ends of the second coil 62 and vice versa. Thus, a ball-shape is formed. In some embodiments, each turn the first coil 60 overlaps the previous turn of the second coil 62, creating overlapping and underlapping coil turns amongst the coils 60, 62.

Similarly, FIGS. 19A-19C illustrate an isolation device 10 formed from individual coils, wherein the device 10 includes a cover 50. FIG. 19A illustrates a first coil 64 positioned horizontally and FIG. 19B illustrates a second coil 66 positioned vertically, wherein the second coil 66 includes a cover 50. In this embodiment, the cover 50 covers one end of the second coil 66. However, it may be appreciated that the cover 50 may cover any portion of the second coil 66. Likewise, more than one cover 50 may be present, and one or more covers 50 may be alternatively or additionally cover portions of the first coil 64. In this embodiment, each of the coils 64, 66 vary in diameter, varying from smaller near its ends and larger near its center. FIG. 19C illustrates the combination of the first coil 64 and second coil 66 forming a ball-shaped body 12. By positioning the coils 64, 66 perpendicularly to each other, the larger center of the first coil 60 engages the smaller ends and cover 50 of the second coil 62 and vice versa. Thus, a ball-shape is formed including a cover 50. Further, the cover 50 may be held in place by sandwiching between the first and second coils 64, 66.

In some embodiments, an isolation device 10 comprised of separate parts is formed into its desired shape, such as a ball-shape, and then delivered to a target location with the body. However, in other embodiments, the separate parts are delivered individually to the target location form the isolation device 10 in vivo. For example, FIGS. 20A-20C illustrate such delivery of the isolation device 10. FIG. 20A illustrates delivery of the first coil 60 (of FIG. 18A) to a target location within a bifurcated blood vessel BV near an aneurysm A. The coil 60 is delivered from a delivery catheter 68 and positioned near the aneurysm A. FIG. 20B illustrates delivery of the second coil 62 (of FIG. 18B) to the target location. The second coil 62 is delivered from the delivery catheter 68 (or from another delivery catheter or device) in an orientation so as to combine with the first coil 60 forming an isolation device 10. In this embodiment, the second coil 62 is delivered at a substantially perpendicular angle to the first coil 60 forming a ball-shaped body 12, as illustrated in FIG. 20C.

Devices for Occluding Endoleaks of Aneurysms

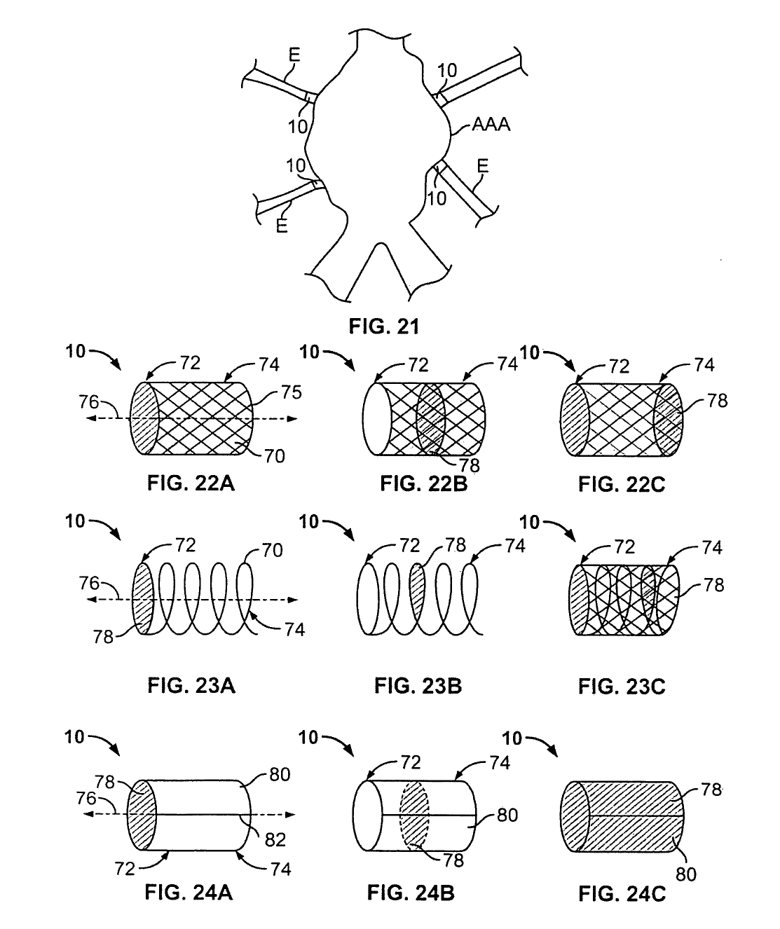

A variety of isolation devices are provided for treating endoleaks of aneurysms, particularly abdominal aortic aneurysms. It may be appreciated that such isolation devices may also be used to occlude any blood vessels within the body or any luminal anatomy. FIG. 21 illustrates an abdominal aortic aneurysm AAA having endoleaks E. Isolation devices 10 of the present invention are shown positioned within the endoleaks E so as to occlude the endoleaks E.

FIG. 22A illustrates an isolation device 10 comprising a body 70 having a first end 72, a second end 74 and a lumen 75 having a longitudinal axis 76 extending therethrough. The isolation device 10 also includes an occluder 78 which occludes blood flow in at least one direction. In this embodiment, the occluder 78 is located near the first end 72 occluding blood flow through the lumen 75 along the longitudinal axis 76 so as to act as an axial occluder. In some embodiments, the isolation device of FIG. 22A has similarities to the isolation device of FIG. 3. However, in this embodiment, the isolation device 10 is configured to be positioned within an endoleak E so as to occlude blood flow in an axial direction.

The body 70 may have any suitable shape or design, such as a cylindrical shape as shown. Further, the body 70 may be comprised of any suitable construction, such as braid, mesh, lattice, coil, struts or other construction. The body 70 shown in FIG. 22A has a wire braid construction. Likewise, the occluder 78 may have any suitable shape, design or construction. For example, the occluder 78 may be comprised of a solid sheet, a sheet having openings, a mesh, a lattice, struts, threads, fibers, filaments, a biocompatible filler or adhesive, or other suitable material. The occluder 78 shown in FIG. 22A comprises a, solid sheet extending across the first end 72. It may be appreciated that the occluder 78 may alternatively extend across the lumen 75 at any position between the ends 72, 74, as illustrated in FIG. 22B. Or, the occluder 78 may encase or encapsulate the body 70, as illustrated in FIG. 22C. In some embodiments, the sheet is comprised of ePTFE and is sandwiched between portions of the body 70 or is bound to a layer of the body 70.

FIGS. 23A-23C illustrate another embodiment of an isolation device 10. Here the body 70 has the form of a coil. Again the body 70 has a first end 72 and a second end 74. The device 10 also includes an occluder 78 located near the first end 72. In some embodiments, the isolation device of FIG. 23A has similarities to the isolation device of FIG. 4. However, in this embodiment, the isolation device 10 is configured to be positioned within an endoleak E so as to occlude blood flow in an axial direction. It may be appreciated that the occluder 78 may alternatively extend across the coil at any position between the ends 72, 74, as illustrated in FIG. 23B. Or, the occluder 78 may encase the body 70, as illustrated in FIG. 23C.

FIGS. 24A-24C illustrate an isolation device 10 constructed from a sheet 80. The sheet 22 is joined, coupled or overlapped along an edge 82 so as to form the body 70 of the device 10 having a cylindrical shape. FIG. 24A illustrates the device 10 having an occluder 78 disposed near the first end 72. It may be appreciated that the occluder 78 may alternatively extend across the device 10 at any position between the ends 72, 74, as illustrated in FIG. 24B. Or, the occluder 78 may encase the body 70, as illustrated in FIG. 24C.

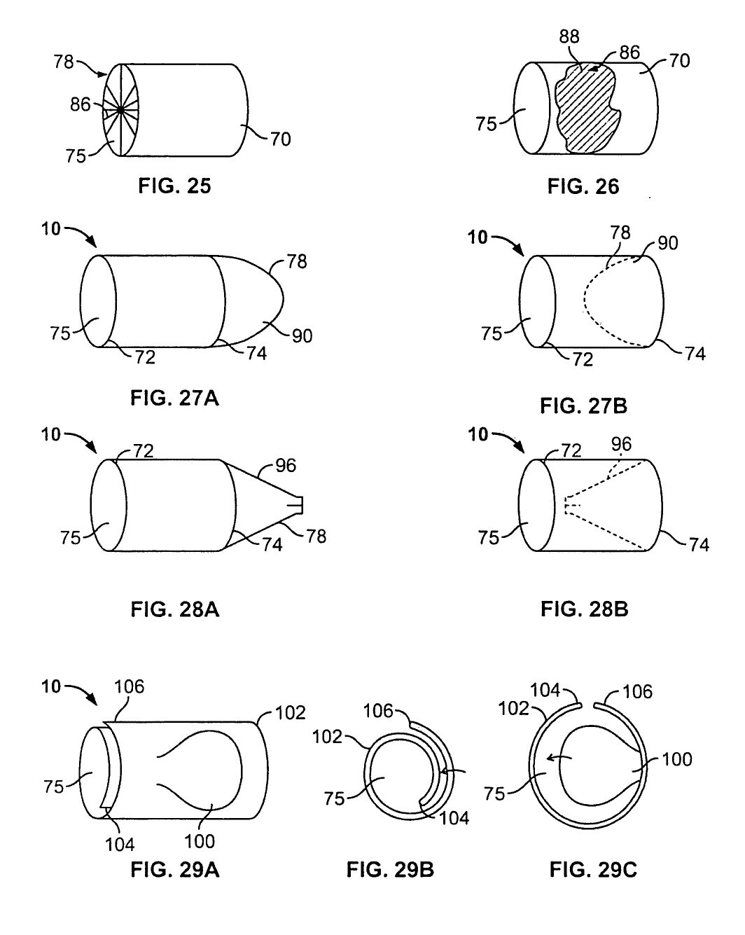

As mentioned, the body 70 may be comprised of any suitable construction, such as braid, mesh, lattice, coil, struts or other construction, and the occluder 78 may have any suitable shape, design or construction, such as a solid sheet, a sheet having openings, a mesh, a lattice, struts, threads, fibers, filaments, a biocompatible filler or adhesive, or other suitable material. FIG. 25 illustrates an occluder 78 comprising fibers 86 that extend across the lumen 75 of the body 70. The fibers 86 may only partially cover the lumen 75, however such coverage may be sufficient to occlude blood flow therethrough. Likewise, the fibers 86 may initiate and encourage thrombus formation to form a more complete seal at a later time. FIG. 26 illustrates an occluder 78 comprising a biocompatible filler 88.

FIGS. 27A-27B illustrate an isolation device 10 having an occluder 78 comprising a sack 90. The sack 90 may be comprised of any flexible material such as ePTFE, urethane or other elastic or polymeric material. FIG. 27A illustrates the sack 90 extending beyond the second end 74 of the device 10. Such a configuration would be typical in situations wherein blood would enter the lumen 75 through the first end 72 moving toward the second end 74. FIG. 27B illustrates the sack 90 extending into the lumen 75. Such a configuration would be typical in situations wherein blood would enter the lumen 75 through the second end 74 moving toward the first end 72.

FIGS. 28A-28B illustrate an isolation device 10 having an occluder 78 comprising a valve 96. The valve 96 typically comprises a one-way valve, such as a duck bill valve. FIG. 28A illustrates the valve 96 extending beyond the second end 74 of the device 10. Such a configuration would be used to block flow of blood which naturally flows from the second end 74 toward the first end 72. Thus, the valve 96 would restrict or prevent flow through the lumen 75. FIG. 28B illustrates the valve 96 extending into the lumen 75. Such a configuration would be used to block flow of blood which naturally flows from the first end 72 toward the second end 74.

FIGS. 29A-29C illustrate an isolation device 10 having an occluder 78 comprising a flap 100. Here the isolation device 10 has a body 70 constructed from a sheet 102 having a first edge 104 and a second edge 106. The sheet 102 is rollable so that the first edge 104 overlaps the second edge 106, as illustrated in FIGS. 29A-29B. In this embodiment, the flap 100 is cut or formed from the sheet 102, and the flap 100 is preformed so as to be biased inward toward the lumen 75. In other embodiments, the flap 100 is attached to the sheet 102. Referring to FIG. 29A, the sheet 102 may be rolled so that portions of the sheet 102 near the first edge 104 overlap the flap 100, thereby supporting the flap 100 and resisting movement of the flap 100 inwardly. FIG. 29B provides an end view of the sheet 102 wherein the flap 100 is resisted from moving inwardly by the portion of the sheet near the first edge 104. In this collapsed configuration, the device 10 is deliverable to a target location in the body. Referring to FIG. 29C, the device 10 may then be deployed, allowing the sheet 102 to unroll so that the first edge 104 and second edge 106 are drawn closer together. This reveals the flap 100 and allows inward movement of the flap 100 to occlude the lumen 75. The flap 100 may be coated or constructed from a material that provides a good seal.

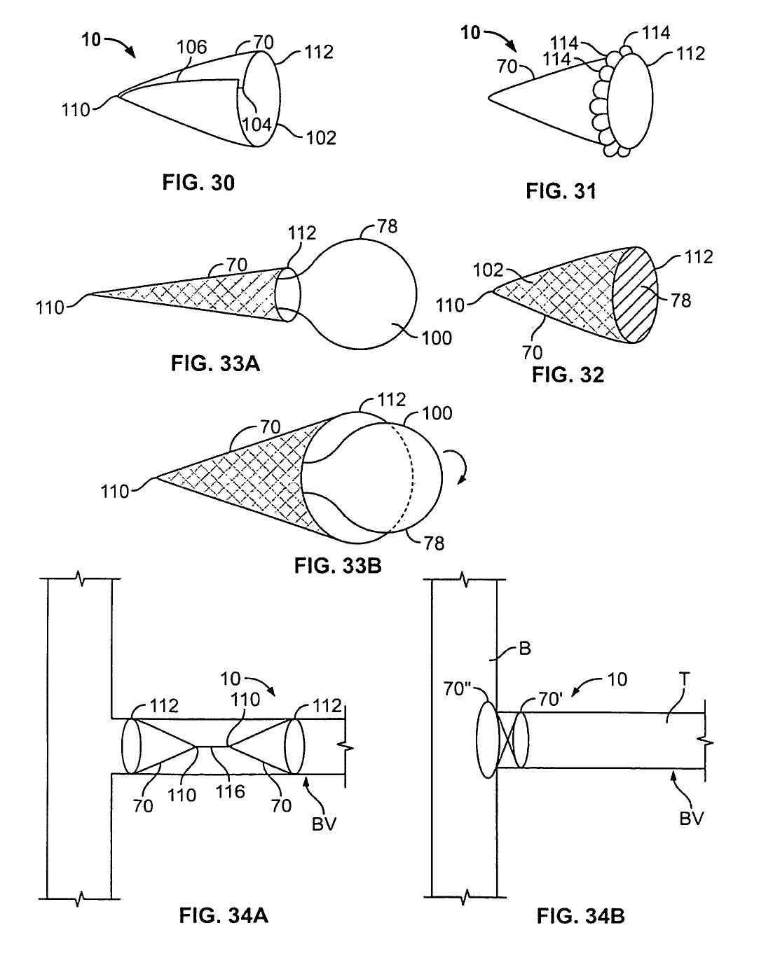

In some embodiments, the isolation device 10 has a conical shaped body 70. FIG. 30 illustrates a device 10 having a body 70 formed from a sheet 102 having a first edge 104 and a second edge 106, wherein the edges 104, 106 meet or overlap so that the body 70 has a conical shape with a tip 110 and a base 112. Thus, the tip 110 forms the occluder by preventing blood flow through the device 10 when the base 112 is expanded within a blood vessel. Optionally, as illustrated in FIG. 31, the base 112 may include anchoring elements 114, such as rings, to assist in anchoring the base 112 to the blood vessel.

In some embodiments, as illustrated in FIG. 32, the conical shaped body 70 is formed from a lattice or mesh sheet 102. In such embodiments, the tip 110 may act as an occluder. However, the device 10 may include an additional occluder 78 over the base 112 to assist in blockage of blood flow therethrough. Similarly, as illustrated in FIGS. 33A-33B, the occluder 78 may be comprised of a biased flap 100 which extends from the base 112 when the body 70 is collapsed (FIG. 33A) and moves inwardly so as to cover the base 112 when the body 70 is expanded (FIG. 33B).

It may be appreciated that in some embodiments, the isolation device 10 is comprised of a plurality of conical shaped bodies 70. FIG. 34A illustrates a pair of conical shaped bodies 70 positioned within a blood vessel BV. As shown, each body 70 has a tip 110 and the tips 110 are coupled, such as by a connector 116, so that the bases 112 face away from each other. Such plurality of bodies 70 may increase the ability of occluding the blood vessel BV. FIG. 34B illustrates alternative positioning of the isolation device 10 of FIG. 34A. Here, the device 10 is positioned so that a first conical shaped body 70′ is positioned within a trunk T of the blood vessel BV and a second conical shaped body 70″ connected directly thereto is positioned at least partially outside of the trunk T, such as within a branch B of the blood vessel BV. Such positioning may also increase the ability of occluding the blood vessel BV.

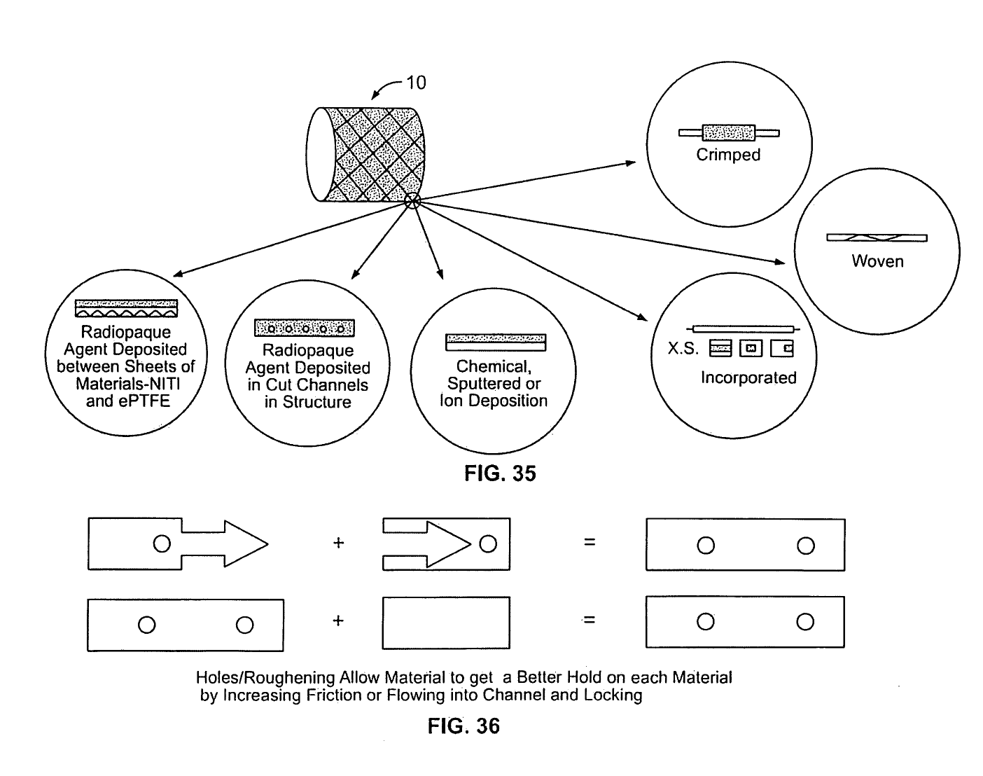

Each of the isolation devices 10 of the present invention may be radiopaque to assist in visualization during placement within a target location in the body. Thus, radiopaque material, such as gold, platinum, tantalum, or cobalt chromium, to name a few, may be incorporated into the device 10. FIG. 35 illustrates a variety of methods of incorporating radiopaque material, such as deposition between sheets of materials (such as nitinol and ePTFE), deposition in cut channels in body of device, chemical deposition, sputtered deposition, ion deposition, weaving, and crimping, to name a few.

In some embodiments, it may be desired to have some components elastic and some inelastic. It is often the case that these materials cannot be easily connected. FIG. 36 illustrates a method where two such materials can be joined by way of a mechanical fit and then sealed by a pressure fit of a material constraining the surface and keeping the dissimilar pieces locked in position relative to each other. This is only an example and many others are possible with a similar objective.

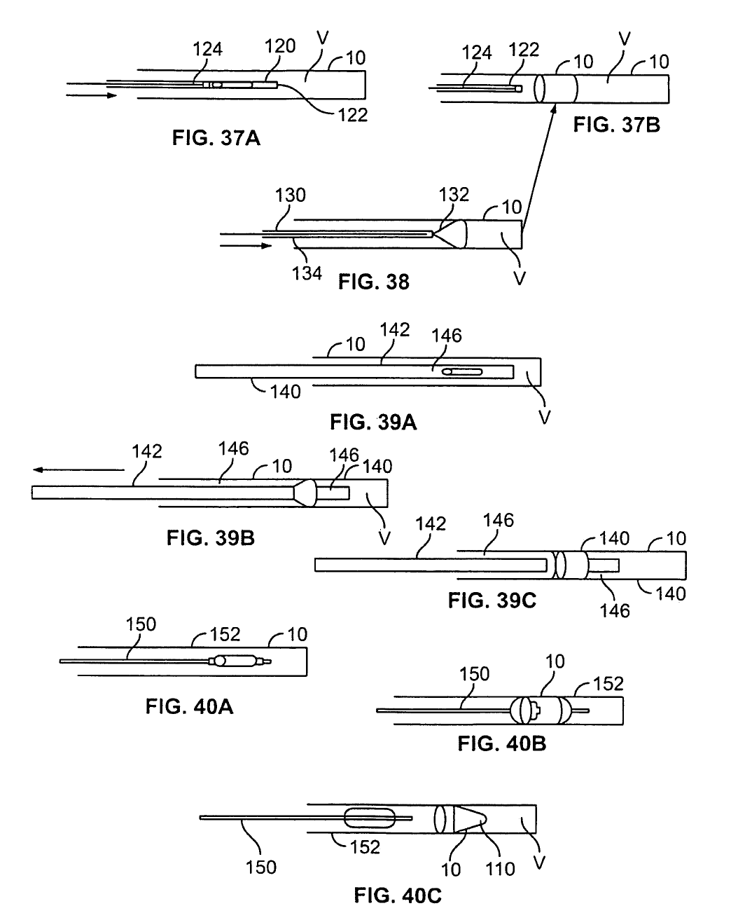

A variety of delivery devices may be used to deliver the isolation devices 10 of the present invention. For example, FIGS. 37A-37B illustrate a push-style delivery system. In this embodiment, the delivery system comprises a catheter 120 having a lumen 122 and a push-rod 124 extending through the lumen 122. The isolation device 10 is loaded within the lumen 122 near the distal end of the catheter 120. The catheter 120 is then advanced through the vasculature to a target delivery site within a blood vessel V. The isolation device 10 is then deployed at the target delivery site by advancing the push-rod 124 which pushes the device 10 out of the lumen 122 and into the blood vessel V.

FIG. 38 illustrates a pull-style delivery system. In this embodiment, the delivery system comprises a catheter 130 having a lumen 132 and a pull element 134 extending through the lumen 132. The isolation device 10 is loaded within the lumen 132 near the distal end of the catheter 130 and attached to the pull element 134. The catheter 130 is then advanced through the vasculature to a target delivery site within a blood vessel. The isolation device 10 is then deployed at the target delivery site by advancing the pull element 134 which pulls the device 10 out of the lumen 132 and into the blood vessel V. It may be appreciated that the pull element 134 may alternatively extend along the exterior of the catheter 130 or through a lumen in the wall of the catheter 130.

FIGS. 39A-39C illustrate a sheath-style delivery system. In this embodiment, the delivery system comprises a rod 140 positionable within a sheath 142. The isolation device 10 is mountable on the rod 140 and the sheath 142 is extendable over the isolation device 10, as illustrated in FIG. 39A. The system is then advanced so that the device 10 is desirably positioned with a blood vessel V. In this embodiment, the rod 140 includes radiopaque markers 146 to assist in such positioning. The sheath 142 is then retracted, as illustrated in FIG. 39B, releasing device 10 within the blood vessel V. Once the device 10 is deployed, as illustrated in FIG. 38C, the rod 140′ may then be retracted leaving the device 10 in place. This type of delivery system may be particularly suited for delivery of devices such as illustrated in FIGS. 27A-27B and FIGS. 28A-28B.

FIGS. 40A-40C illustrate a balloon expandable delivery system. In this embodiment, the delivery system comprises a catheter 150 having an expandable balloon 152 mounted near its distal end. The isolation device 10 is crimped over the balloon 152 as illustrated in FIG. 40A. The catheter 150 is advanceable so that the device 10 may be positioned at a target location within a blood vessel V. The balloon 152 may then be expanded (FIG. 40B) which in turn expands the device 10, securing the device 10 within the blood vessel. In this embodiment, the device 10 has a conical shape wherein the tip 110 comprises an elastic material which allows the tip 110 to recoil after delivery, as shown in FIG. 40C. This type of delivery system may be particularly suited for delivery of devices such as illustrated in FIGS. 29A-29C and FIGS. 33A-33B.

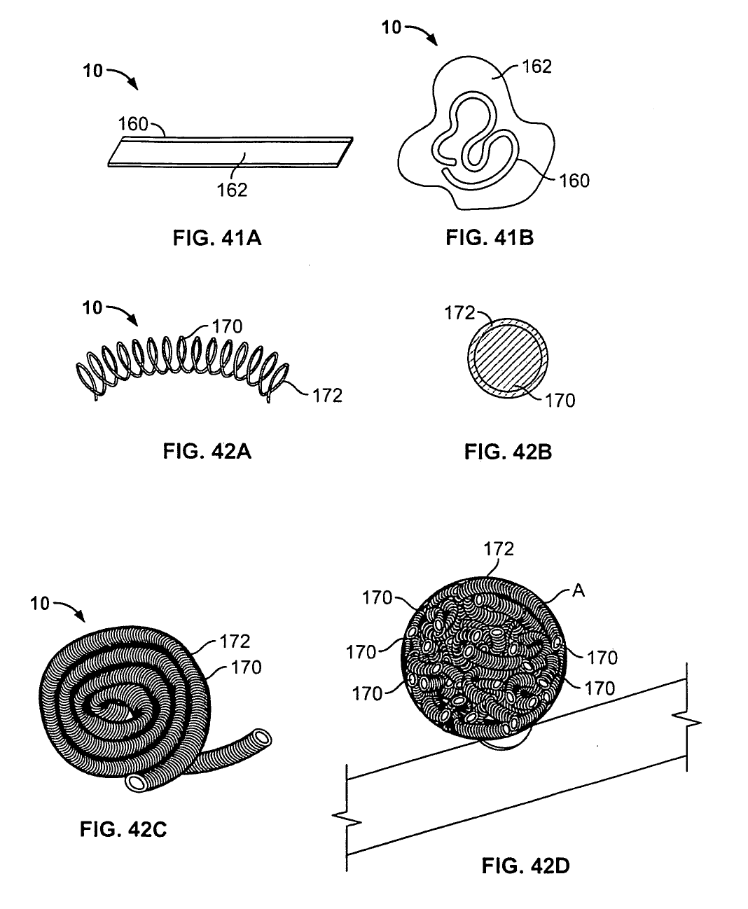

FIGS. 41A-41B illustrate another embodiment of an isolation device 10. In this embodiment, the isolation device 10 comprises a shape memory element 160, such as a wire or ribbon comprised of nitinol, coupled with a portion of material 162, such as a sheet or ribbon comprised of ePTFE. The shape memory element 160 is attached the portion of material 162, such as along an edge as shown in FIG. 41A. When the shape memory element 160 has a linear configuration, the device 10 may be loaded into a lumen of a delivery catheter or delivery device for advancement to a target location within a blood vessel. The shape memory element 160 may then change shapes to a curled, coiled, or random shape, causing the isolation device 10 to form a ball-shape as illustrated in FIG. 41B. The ball-shape thus occludes flow through the blood vessel at the target location.

FIGS. 42A-42D illustrate another embodiment of an isolation device 10. In this embodiment, the isolation device 10 comprises a coil 170 having a heat activated covering 172. The coil 170 may comprise a conventional embolic coil, such as a Guglielmi Detachable Coil (GDC). A GDC is a platinum alloy or similar coil, which has a natural tendency or a memory effect, allowing it to form a coil of a given radius and coil thickness and softness. GDC coils are manufactured in a variety of sizes from 2 mm in diameter or more, and in different lengths. Further, conventional GDCs are available in a variety of coil thicknesses, including 0.010″ and 0.018″, and two stiffnesses (soft and regular). It may be appreciated that the coil 170 may alternatively be comprised of other types, sizes and materials.

FIG. 42B provides a cross-sectional view of the coil 170 having the covering 172. Example coverings 172 include thermoplastic materials and thermoplastic elastomers, such as polyurethane, polyester, Pebax B, nylon, pellathane, TecoflexB and TecothaneB. Example coverings 172 also include heat activated adhesives. One or more coils 170 are then delivered to a blood vessel, such as an endoleak. Once delivered, the coil 170 is heated up, allowing the covering 172 to reach a glass-transition temperature and turning it into a soft semi-gelatinous consistency. Upon cooling, the covering 172 reforms its shape, acting as a glue or binding agent. A single coil 170 which has been heated and cooled will hold its three-dimensional shape as shown in FIG. 42C, making it more stable for occluding the blood vessel.

Such coils 170 may also be used to treat berry aneurysm. In such instances, a catheter is advanced into the blood vessel supplying the aneurysm. A second smaller catheter called a microcatheter is then advanced through the catheter to the aneurysm. The coils 170 are placed through the microcatheter into the aneurysm until the aneurysm is satisfactorily filled. Multiple coils 170 packed into an aneurysm A (FIG. 42D) will become “locked together as the covering 172 binds to the neighboring coil. Each coil 170 is typically heated as it is delivered, such as by using the delivery catheter to input radiofrequency energy. Alternatively, all of the coils 170 could be heated at the same time, such as with a secondary radiofrequency induction catheter, or with an external MRI field.

The thickness of the covering 172 could be adjusted for optimum performance. The coils shown in FIG. 42D are locked together by the heated and cooled covering, causing the coils to resist further re-packing or remodeling. Typically, conventional GDC coils (without such a polymeric covering) are not stable within the aneurysm and can rearrange shape, position and packing density leading to reduce effectiveness. In some instances, intervention in needed to add additional coils to improve packing. However, the covering 172 of the present invention resists further re-packing or remodeling. The covering 172 could also aid in reducing the free space between coils 170.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended.

What is claimed is:

1. An isolation device for isolating an aneurysm located near an intersection of a trunk of a blood vessel and a plurality of branches, the isolation device comprising:
a body having a first end, a second end and a lumen therethrough along a longitudinal axis, wherein the body is configured for positioning within the intersection so that the aneurysm is substantially aligned with the longitudinal axis;
an occluder disposed across the lumen so as to allow blood flow into the first end along the longitudinal axis and to divert flow away from the longitudinal axis so as to isolate the aneurysm.

2. An isolation device as in claim 1, wherein the body includes a body wall extending between the first and second ends, and wherein the wall has openings configured to facilitate the diversion of flow away from the longitudinal axis.

3. An isolation device as in claim 2, wherein the body wall comprises a mesh having smaller openings arranged to facilitate flow through along the longitudinal axis near the first end and larger openings arranged to facilitate the diversion of flow away from the longitudinal axis.

4. An isolation device as in claim 1, wherein the occluder comprises a solid sheet, a sheet having openings, a lattice, struts, threads, fibers, filaments, a biocompatible filler, an adhesive or a combination of these.

5. An isolation device as in claim 4, wherein the occluder comprises a diverter having a conical shape and a tip, wherein the tip faces the first end so as to facilitate diversion of flow away from the longitudinal axis.

6. An isolation device as in claim 1, wherein the occluder comprises a sack extendable from the second end into the aneurysm.

7. An isolation device as in claim 1, wherein the body has a coiled shape.

8. An isolation device as in claim 1, wherein the first end is configured to anchor the isolation device within the trunk and wherein the second end is configured to atraumatically reside near a neck of the aneurysm.

9. An isolation device for isolating an aneurysm located near an intersection of a trunk of a blood vessel and a plurality of branches, the isolation device comprising: a body having a ball shape, wherein the body is configured for positioning within the intersection so as to allow blood flow through the trunk to the plurality of branches while diverting flow away the aneurysm.

10. An isolation device as in claim 9, wherein the body comprises a braid, mesh, lattic, coil, strut, or combination of these.

11. An isolation device as in claim 10, wherein the body comprises articulating struts.

12. An isolation device as in claim 9, further comprising a cover disposed over a portion of the body, wherein the cover is configured to divert flow away from the aneurysm.

13. An isolation device as in claim 9, wherein the body is comprised of separate parts that together form the ball shape.

14. An isolation device comprising: at least one body having a covering, wherein the covering is transitionable from non-binding state wherein contacting portions of the body are able to move relative to each other at each contact point and a binding state wherein the contacting portions of the body are restricted from moving relative to each other at each contact point.

15. An isolation device as in claim 14, wherein the body has a coil shape.

16. An isolation device as in claim 14, wherein the covering is transitionable in response to heat.

17. An isolation device as in claim 16, wherein the covering is transitionable in response to radiofrequency energy.

18. An isolation device as in claim 16, wherein the covering comprises a polymer.
